(19) United States
(12) Patent Application Publication
(10) Pub. No.: US 2011/0015267 A1
(43) Pub. Date: Jan. 20, 2011

(54) METHODS FOR THE PREPARATION AND USE OF AQUEOUS SOLUTIONS OF MAGNESIUM VALPROATE HYDRATE AND L-CARNITINE

(76) Inventor: Deanna Jean Nelson, Raleigh, NC (US)

Correspondence Address: Casimir Jones, S.C., 2275 DEMING WAY, SUITE 310, MIDDLETON, WI, 53562, US

(21) Appl. No.: 12/505,849

(22) Filed: Jul. 20, 2009

(51) Int. Cl. A61K 31/22

(52) U.S. Cl. 514/546

(57) ABSTRACT

The present invention relates to methods for preparing liquid valproate compositions comprising solutions of a magnesium valproate and L-carnitine in water and administering said compositions to a subject in need of treatment with valproate and L-carnitine. The compositions of the invention are particularly useful treatments for infants, young children, women of child-bearing age, the aging, institutionalized patients, and patients having dysphagia who are receiving valproate treatment and are at high risk for valproate-related L-carnitine deficiency and cellular dysfunction and hepatotoxicity related thereto.

### FIELD OF THE INVENTION

The present invention relates to methods of preparing palatable liquid valproate compositions comprising solutions of a magnesium valproate salt and L-carnitine in water and administering said compositions to a subject in need of treatment with valproate and L-carnitine. Pharmaceutical compositions are also provided that are useful therapies for the treatment of neurological, immunological, and retroviral-mediated disorders in warm-blooded mammals. The compositions of the invention are particularly useful treatments for infants, young children, women of child-bearing age, the aging, institutionalized patients, and patients having dysphagia who are receiving valproate treatment and are at high risk for valproate-related L-carnitine deficiency and cellular dysfunction and hepatotoxicity related thereto.

### BACKGROUND OF THE INVENTION

Valproic acid is a branched carboxylic acid having the molecular formula C₈H₁₆O₂. Valproic acid is also known as 2-propylpentanoic acid, 2-propylvaleric acid, and dipropylacetic acid. Valproic acid is a colorless liquid having a boiling point of 120-121° C. at 14 torr. The compound is very slightly soluble in water. It has a pKa of 4.6, and reacts with bases to form salts generally known as valproates.

“Valproate,” a term which encompasses both valproic acid and valproate salts, has been approved by regulatory agencies around the world, including the U.S. Food and Drug Administration (FDA), for more than thirty years. Valproate is a therapy for several clinical indications, including neurological disorders, mania, manic episodes associated with bipolar disorder, epilepsy, and affective and attention deficit disorders. In addition, valproate is used for the prophylactic treatment, modulation and management of migraine headache, chronic pain, and neuropathic pain. Further, potential therapeutic benefits of valproate in still other clinical indications are being evaluated in on-going clinical trials. Valproate therapy is being evaluated in clinical studies assessing activity of the substance as a histone deacetylase inhibitor to promote cell differentiation and regeneration, or to regulate gene expression in subjects afflicted with spinal muscular atrophy. Likewise, valproate may exhibit therapeutic benefit as a combinatorial therapeutic treatment of human cancers and for the treatment of tumor metastasis. Similarly, valproate may be useful in the treatment and management of pain, for treating severe tinnitus, for treatment of disorders of personal attachment and deficient social interaction, or for treating Alzheimer's disease. Pre-clinical studies also show that valproate may promote neural stem cell differentiation and or be useful as a co-medicament to promote the elimination of the Human Immunodeficiency Virus (HIV) or other retroviruses from the body or to prevent progression of a retroviral infection to AIDS.

Today, most valproate is provided as solid dosage forms (e.g., tablets or capsules) of divalproex sodium (Depakote, Abbott Laboratories, Inc.). A small percentage of patients receive either valproic acid or sodium valproate as solid dosage forms or sodium valproate as solid and liquid dosage forms. In general, valproate contained in all conventional dosage forms is rapidly and nearly completely absorbed from the gastrointestinal tract with peak plasma concentrations occurring 1-4 hours after ingestion. Therapeutic doses of valproate range from 1 to 2 g daily for adults and 15 to 60 mg/kg in children, with the goal of achieving therapeutic serum concentrations of from 50 to 125 μg/mL.

The drug is usually well tolerated. However, rare serious complications may occur in some patients receiving valproate chronically, including fatal hemorrhagic pancreatitis, bone marrow suppression, hepatotoxicity and hyperammonemic encephalopathy. In addition, infants, young children, women of child-bearing age, the aging, institutionalized patients, and patients having dysphagia who are receiving valproate treatment are at high risk for valproate-related L-carnitine deficiency and cellular dysfunction and hepatotoxicity related thereto.

In humans and other mammals, valproate is metabolized extensively in the liver by direct glucuronidation (80%) and by a combination of β- and ω-oxidations in mitochondria and the cytosol, respectively (17%); 3% of valproate is excreted in the urine as an L-carnitine ester which is not re-adsorbed by the kidney as is free carnitine. Mitochondrial β-oxidation generates short-chain fatty acids such as 3-hydroxy-2-propylpentanoic acid, 2-propyl-2-pentenoic acid, and 2-propyl-2,4-pentadienoic acid; both α,β-unsaturated fatty acids have been identified in the literature as a genotoxic substance incriminated in the development of cerebral edema and in the hepatotoxicity of valproate. Cytosolic ω-oxidation generates short-chain fatty acids that contain ω-hydroxyl groups, ω1-hydroxyl groups, or α,β-unsaturated fatty acids that have been identified in the literature as genotoxic substances (2-propyl-4-pentenoic acid and 2-propyl-3-pentenoic acid) that could also promote hyperammonemia. (During long-term valproate therapy, a greater degree of ω-oxidation occurs, potentially increasing the risk for toxicity.)

It has been known for over twenty-five years that valproate interferes with L-carnitine metabolism and may cause L-carnitine deficiency. [Ohtani Y, Endo F, Matsuda I. Carnitine deficiency and hyperammonemia associated with valproic acid therapy. J Pediatr 1982 November; 101(5): 782-785.] L-Carnitine deficiency is a serious disorder that is frequently diagnosed in children, pregnant women, the aging, and institutionalized patients who are receiving valproate therapy. Further, in some cases, valproate can cause a severe or fatal hepatotoxicity, particularly in young children. Valproate-related hepatotoxicity is also known to be related to interference with L-carnitine metabolism.

Other mechanisms of valproate-induced toxicity include inhibition of L-carnitine synthesis, glutathione depletion, direct inhibition of specific enzymes in the fatty acid oxidation sequence by CoA esters of unsaturated valproate metabolites, inhibition of plasmalemmal carnitine transporter, and promotion of transport of glutamine through the mitochondrial membrane, thereby enhancing glutaminase activity and release of ammonia. Indirect effects resulting from valproate-related carnitine deficiency include adverse alterations in the activity of carnitine-dependent long-chain acyltransferases, including acyltranferases involved in neuronal triglyceride and phospholipid fatty acid turnover.

Mitochondrial β-oxidation of long-chain fatty acids involves their transport within the mitochondrial matrix using a pathway often called the “carnitine shuttle.” The carnitine shuttle consists of the following steps. First, in the cytosol, a fatty acid is activated by enzyme-catalyzed linkage to coenzyme A (CoASH) to form a fatty acyl-CoA. The fatty acyl-CoA crosses the outer mitochondrial membrane. Palmitoyl carnitine transferase (PCT1) then catalyzes the transfer of the fatty acid from CoA to carnitine; this step is needed because the inner mitochondrial membrane is not permeable to long-chain or branched acylcarnitines. The fatty acylcarnitine is then exchanged for free carnitine by carnitine translocase. In the mitochondrial matrix, a second carnitine transferase, PCT2, catalyzes the exchange between the fatty acylcarnitine and CoASH, providing the fatty acyl-CoA, which enters the fatty acid β-oxidation process. Carnitine-mediated reverse transfers by the same pathway also help prevent fatty acyl-CoA accumulation.

Likewise, mitochondrial β-oxidation of valproate involves its transport within the mitochondrial matrix using the same carnitine shuttle pathway. First, in the cytosol, valproate is activated by enzyme-catalyzed linkage to coenzyme A (CoASH) to form valpropyl-CoA. Valproyl-CoA crosses the outer mitochondrial membrane. Palmitoyl carnitine transferase (PCT1) then catalyzes the transfer of valproate from CoA to carnitine; this step is needed because the inner mitochondrial membrane is not permeable to branched acylcarnitines (valproylcarnitine, for example). Valproylcarnitine is then exchanged for free carnitine by carnitine translocase. In the mitochondrial matrix, a second carnitine transferase, PCT2, catalyzes the exchange between valproylcarnitine and CoASH, providing valproyl-CoA, which enters a slow β-oxidation process. Carnitine-mediated reverse transfers by the same pathway also help prevent valproyl-CoA accumulation.

Because carnitine-shuttle mediated transfers of valproate are significantly less efficient than the corresponding transfers of fatty acids, valproylcarnitine and its metabolites accumulate within the mitochondria and cells and tissues of the body. Valproylcarnitine and related metabolites interfere with normal fatty acid metabolism and exhibit other undesirable biological activities not related to fatty acid metabolism.

Clinicians have shown that administration of both valproate and L-carnitine effectively treats both valproate-related L-carnitine deficiency and valproate-related toxicities. [See, for example, the review by Lheureux P E, Hantson P. Carnitine in the treatment of valproic acid-induced toxicity. Clin Toxicol (Phila). 2009 February; 47(2): 101-111 and references therein.]

In European Patent Specification EP 0637,449 B1 Cavazza discloses the use of L-carnitine or an acyl L-carnitine or their pharmacologically acceptable salts and a pharmacologically acceptable salt of valproic acid for producing a medicament for decreasing the seizure frequency in epileptic patients. Cavazza also discloses pharmaceutical compositions for decreasing the seizure frequency in epileptic patients which comprise an acyl L-carnitine or a pharmaceutically acceptable salt thereof and a pharmacologically acceptable salt of valproic acid. Cavazza further discloses pharmaceutical compositions in unit dosage form comprising an amount of acyl L-carnitine or a pharmacologically acceptable salt thereof equivalent to 0.3-0.5 g of L-carnitine and from 0.2 to 0.5 g of sodium valproate or magnesium valproate. Cavazza's invention relates to the coordinated use of L-carnitine, acyl L-carnitines, or their pharmacologically acceptable salts and valproate (i.e., a pharmacologically acceptable salt of valproic acid, such as sodium or magnesium valproate). By “coordinated use” Cavazza means the substantially concomitant supplementation of L-carnitine or acyl L-carnitine or a pharmacologically acceptable salt thereof and valproate, as active ingredients, and the administration of a combination preparation containing a mixture of the aforesaid active ingredients, in addition to suitable excipients, if any.

Infants, young children, women of child-bearing age, the aging, institutionalized patients, and patients having dysphagia who are receiving valproate treatment are at greatest risk for valproate-related L-carnitine deficiency and cellular dysfunction and hepatotoxicity related thereto. Therapeutic doses of valproate range from 1 to 2 g daily for adults and 15 to 60 mg/kg in children, with the goal of achieving therapeutic serum concentrations of from 50 to 125 μg/mL. Typically, unit doses of valproate consist of 250, 500, or 1,000 mg of drug in a tablet or capsule that also contains sizeable quantities of excipients and other inactive formulation components designed to control and/or extend release of the drug into the alimentary tract. As a result, conventional valproate dosage forms are frequently solid dosage forms that are large in size and difficult to swallow. Likewise, conventional L-carnitine dosage forms are solid dosage forms provided as tablets or capsules that contain 250, 500, or 1,000 mg of L-carnitine and sizeable quantities of excipients. Because L-carnitine is unstable in these dosage forms, it releases a repugnant odor and has a repugnant taste, factors which increase difficulty in ingesting and swallowing conventional L-carnitine dosage forms.

To date, however, no liquid pharmaceutical formulations comprising palatable aqueous solutions having quantitatively precise defined proportions of valproate and L-carnitine have been provided to meet the especial therapeutic needs of the individuals such as those disclosed above who have difficulty in ingesting and swallowing large dosage forms which may smell or taste repugnant. The present invention remedies these shortcomings.

### DETAILED DESCRIPTION OF THE INVENTION

The present invention relates to methods for preparing palatable liquid dosage forms comprising aqueous solutions of a magnesium valproate salt and L-carnitine inner salt, wherein the magnesium valproate salt is magnesium valproate hydrate or L-carnitine magnesium valproate. Both magnesium valproate hydrate and L-carnitine magnesium valproate are water-soluble, bioavailable sources of valproate, a drug that is useful for several clinical indications, including neurological disorders, including mania, manic episodes associated with bipolar disorder, epilepsy, and affective and attention deficit disorders, and the prophylactic treatment, modulation and management of migraine headache, chronic pain, and neuropathic pain. L-Carnitine inner salt is a water-soluble, bioavailable source of L-carnitine, a molecule that mediates trafficking of fatty acids across cellular and mitochondrial membranes. The liquid dosage forms of the invention provide palatable combinations of a magnesium valproate salt and L-carnitine in water that provide both valproate and L-carnitine in a dosage form that is easily administered to individuals who require valproate therapy and are at risk for L-carnitine deficiency.

The present invention also relates to a method of formulating palatable liquid dosage form comprising a magnesium valproate salt and L-carnitine inner salt in water or aqueous solutions in concentrations that provide a therapeutically effective amount of valproate and a therapeutically effective amount of L-carnitine to a warm-blooded animal after the formulation is administered to the animal.

The present invention provides liquid pharmaceutical formulations having defined proportions of valproate and L-carnitine useful for the concomitant provision of definite quantities of valproate and L-carnitine. The liquid pharmaceutical formulations having defined proportions comprise compositions made up of a first known quantity of valproate as magnesium valproate hydrate or L-carnitine magnesium valproate and a second known quantity of L-carnitine as L-carnitine inner salt in water in doses having a first therapeutically effective quantity of valproate and a second therapeutically effect quantity of L-carnitine.

Magnesium valproate hydrate, a magnesium salt of valproic acid, is a white solid having the molecular formula C₁₆H₃₀O₄Mg.xH₂O, where x is about 0.7, and structural formula I:

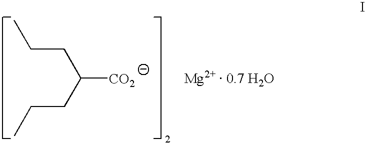

By weight, its composition is 59.4% carbon, 9.8% hydrogen, 7.5% magnesium, and 23.3% oxygen. Magnesium valproate hydrate is 88.6% valproate by weight. Preparation and use of magnesium valproate hydrate is disclosed in U.S. Pat. No. 7,482,486 B2 (Nelson et al.). Nelson discloses a method for the preparation of magnesium valproate hydrate having a definite composition and purity (i.e., freedom from contaminating inorganic salts and insoluble materials), properties that are required for pharmaceutical formulation. Further, magnesium valproate hydrate is a bioavailable valproate salt. In other words, magnesium valproate hydrate is a salt of valproic acid that provides the drug valproate in a readily water-soluble form that is physiologically useful and therapeutically beneficial.

L-carnitine magnesium valproate is a second bioavailable valproate salt. Like magnesium valproate hydrate, L-carnitine magnesium valproate is a salt of valproic acid that provides the drug valproate in a form that is physiologically useful and therapeutically beneficial. L-Carnitine magnesium valproate is a white solid having the molecular formula C₁₆H₃₀O₄Mg and structural formula II:

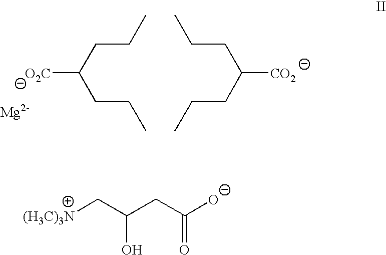

By weight, its composition is 59.4% carbon, 9.8% hydrogen, 7.5% magnesium, and 23.3% oxygen. L-Carnitine magnesium valproate is about 88.6% valproate by weight. Preparation and use of L-carnitine magnesium valproate is disclosed in U.S. Pat. No. 7,456,216 B2.

L-Carnitine inner salt (CAS Registry No. 541-14-0) is a physiologic compound having the molecular formula C₇H₁₅NO₃ and structural formula III:

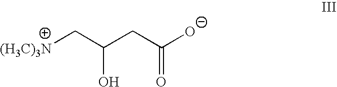

L-Carnitine inner salt is commercially available.

The term “defined composition” as used herein means that a chemical has a definite chemical composition that conforms to theoretical expectations. The definite chemical composition can be determined by analysis. Further, the fact that a chemical has a definite chemical composition indicates that the chemical can be prepared in a consistent and reproducible manner having a purity in the range of 97%-102%. A chemical that meets this criterion can be used to prepare pharmaceutical formulations having quantitatively precise defined proportions of the chemical and its dissociable subunits, if any dissociable subunits are present.

The term “excipient material” means any compound forming a part of the formulation, which is not intended to have independent biological activity, and which is added to a formulation to provide specific characteristics to the dosage form, including providing protection to the active ingredient from chemical degradation, adjusting viscosity, adding colors or flavors, and so forth.

The terms “treating” and “treatment” and the like are used herein to generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein encompasses any treatment of a disease in a mammal, particularly a human and includes: (a) preventing the disease from occurring in a subject which may be predisposed to the disease but has not yet been diagnosed as having it; (b) inhibiting the disease or arresting its development; or (c) relieving the disease, causing regression of the disease and/or its symptoms or conditions.

The phrase “therapeutically effective” is intended to qualify the amount of magnesium valproate hydrate for use in the orally or intravenously administered therapy which will achieve the goal of providing a biologically available (i.e., bioavailable) concentration of the drug valproate to effect reducing or preventing, for example, a neurological, immunological, or viral-related disorder, while avoiding adverse side effects typically associated with valproic acid, sodium valproate compositions, or other valproate salts. Likewise, the phrase is intended to qualify the quantity of L-carnitine inner salt for use in the orally or intravenously administered therapy which will achieve the goal of providing a biologically available concentration of L-carnitine to avoid adverse side effects typically associated with L-carnitine deficiency.

Included within the scope of this invention is a method of treating neurological disorders, immune disorders, or viral-related disorders in a warm-blooded animal using pharmaceutical compositions comprising a magnesium valproate salt, L-carnitine inner salt, water, and suitable pharmaceutical excipients.

For the purpose of this disclosure, a warm-blooded animal is a member of the animal kingdom which includes but is not limited to mammals and birds. The most preferred mammal of this invention is human.

The inventor investigated methods for the preparation of long-needed, palatable liquid compositions having quantitatively precise defined proportions of valproate and L-carnitine in water. During the investigation, the inventor examined formulations comprising conventional valproates (Table 1) and discovered that these valproates did not provide palatable liquid compositions having quantitatively precise defined proportions of valproate and L-carnitine when combined with L-carnitine inner salt in water. For example, valproic acid was poorly soluble in water and failed to provide clear and colorless solutions of valproate at valproate concentrations sufficiently high to be therapeutically useful. Instead, oily droplets of valproic acid adhered to the formulation vessel, making it impossible to formulate compositions having quantitatively precise defined proportions of valproate. Divalproex sodium decomposed on addition to water, again making it impossible to formulate compositions having quantitatively precise defined proportions of valproate. Magnesium valproate, prepared as disclosed in U.S. Pat. No. 5,180,850, did not dissolve completely in water, indicating that the magnesium valproate of '850 was not pure but was a mixture of magnesium valproate and insoluble or partially soluble, unknown materials. This lack of purity renders it impossible to weigh calculated quantities of the magnesium valproate of '850 that are required for a quantitatively precise defined formulation and formulate a composition having the corresponding quantitatively precise defined proportions of valproate and L-carnitine; the weighed quantities of the magnesium valproate of '850 will contain variable proportions of impurities that are water-soluble, partially water-soluble, and water-insoluble as well as the magnesium valproate, and the resulting formulation will contain less than quantitatively precise proportions of valproate. Sodium valproate rapidly adsorbed water from the atmosphere, making it impossible to weigh known quantities to formulate compositions having quantitatively precise defined proportions of valproate. Potassium valproate, cesium valproate, and rubidium valproate were not tested, because high concentrations of potassium, cesium, and rubidium are toxic.

TABLE 1

| | | Result When Combined With L- |
|---|---|---|
| Valproate | Source | Carnitine Inner Salt in Water |
| Valproic acid | Sigma-Aldrich Chemical Co., | Valproic acid was poorly soluble in |
| | Milwaukee, WI | water and failed to provide clear & |
| | | colorless solutions of sufficiently |
| | | high valproate concentration to be |
| | | useful. |
| Divalproex sodium | Prepared as described in U.S. Pat. No. | Divalproex sodium decomposed on |
| | 4,988,731 | addition to water. |
| Magnesium valproate | Prepared as described in U.S. Pat. No. | Magnesium valproate produced in |
| | 5,180,850 | accordance with U.S. Pat. No. 5,180,850 |
| | | failed to dissolve completely; |
| | | insoluble white solid remained. |
| Sodium valproate | Sigma-Aldrich Chemical Co., | Sodium valproate is hygroscopic |
| | Milwaukee, WI | and absorbed water during |
| | | weighing. Solution was clear & |
| | | colorless but tasted “salty.” |
| Potassium valproate, | As disclosed in U.S. Pat. No. 4,558,070 | Not tested; at daily doses of |
| cesium valproate, | | valproate required for therapeutic |
| rubidium valproate | | benefit, the dose of potassium, |
| | | cesium, or rubidium would be toxic. |
| Calcium valproate | As disclosed in U.S. Pat. No. 3,814,812 | Not tested; calcium valproate has |
| | | been reported to be carcinogenic. |

In contrast to the findings disclosed in Table 1, after further investigations, the inventor discovered methods for the preparation of heretofore unknown but long-needed, palatable liquid compositions of valproate and L-carnitine in water having defined proportions of valproate and L-carnitine. The methods comprise dissolving a first quantity of a pure magnesium valproate and a second quantity of L-carnitine inner salt in water. The pure magnesium valproate is selected from the group consisting of magnesium valproate hydrate and L-carnitine magnesium valproate. These two valproate salts have been selected by the inventor by reason of the fact that each salt has a definite chemical composition, thereby enabling formulation to provide a quantitatively precise defined concentration of valproate in the solution. Further, each salt is water soluble and provides a solution in which the valproate concentration can be adjusted to a level in the range from about 1 mg/mL to about 150 mg/mL. Thus, a pure magnesium valproate of the present invention can be compounded and formulated in quantitatively precise defined proportions to provide formulations comprising quantitatively precise proportions of valproate and L-carnitine. Each salt also provides magnesium, a cation that provides buffering capacity and is a physiologically useful nutrient. When dissolved in water, each salt provides a clear, colorless, and tasteless solution having near neutral pH. The inventor has discovered that L-carnitine inner salt can be added to an aqueous solution of a pure magnesium valproate in quantities sufficient to provide an L-carnitine concentration in the range from about 25 mg/mL to about 1,000 mg/mL. The inventor has selected the L-carnitine inner salt because it is a water-soluble, bioavailable source of L-carnitine, and provides only L-carnitine and no other cations or anions that might alter the organoleptic properties or physiological activity of the valproate or the L-carnitine in the formulation. The resulting composition provides quantitatively precise defined proportions and therapeutically useful concentrations of both valproate and L-carnitine in a palatable, liquid dosage form. Further, the composition provides defined proportions of both valproate and L-carnitine in an aqueous dosage form that is easily administered to individuals who require valproate therapy and are at risk for L-carnitine deficiency. The composition is free-flowing, has a viscosity similar to that of water, and has a neutral pH. These properties distinguish compositions of the invention from compositions prepared using conventional valproates.

The inventor has selected water as the pharmaceutical carrier, because water generally exhibits no adverse physiological effects. (In contrast, liquid formulations containing alcohols or halogenated solvents, by way of example, exhibit adverse physiological effects associated with those solvents.) Further, the inventor recognizes that L-carnitine is a nutrient both for mammals and for microbes. Therefore, aqueous solutions of the invention must be prepared to be free of microbial contaminants. Most preferably, water that has been purified to meet US Pharmacopeial standards is used in preparing aqueous solutions of the invention. [U.S. Pharmacopeia, Volume 32. U.S. Pharmacopeial Convention, Rockville, Md. USP Monograph entitled “Purified Water” and chapter entitled “Water for Pharmaceutical Purposes”<1231>.] Water can be purified in known ways to be free of trace contaminants and microbes that cause undesirable side reactions when administered therapeutically to a subject. However, L-carnitine decomposes when subjected to heating. Therefore, the inventor has used aseptic processing and sterilization by filtration through a filter having a pore-size sufficiently small to allow passage of the composition of the invention through the pores but prevent passage of microbes to prepare compositions for pharmaceutical use.

The inventor has unexpectedly found that a liquid composition of the invention is an odorless and tasteless clear solution that is palatable. In contrast, mixtures of a solid valproate with a solid L-carnitine, one of its pharmaceutically useful salts, or an acyl L-carnitine have a repugnant taste and odor.

### Dosage Forms

In general, the pharmaceutical compositions of this invention can be prepared by conventional techniques, as are described in Remington's Pharmaceutical Sciences, a standard reference in this field [Gennaro A R, Ed. Remington: The Science and Practice of Pharmacy. 20^{th} Edition. Baltimore: Lippincott, Williams & Williams, 2000]. For therapeutic purposes, the active components of this combination therapy invention are ordinarily combined with one or more adjuvants appropriate to the indicated route of administration.

The pharmaceutical compositions of this invention can be administered by any means that effects contact of the active ingredients with the site of action in the body of a warm-blooded animal. For example, the means can be oral or parenteral (i.e., subcutaneous, intravenous, intramuscular or intraperitoneal). Alternatively or concurrently, the means of administration can be by more than one route (e.g., oral and parenteral). A most preferred means of administration is by the oral route (i.e., ingestion).

The active ingredients can be administered by the oral route in liquid dosage forms, such as elixirs, syrups, and suspensions. Liquid dosage forms of a composition of the invention are useful for a number of reasons. For example, the dose can easily be adjusted by dilution. Likewise, an oral liquid form can readily be administered to children or people unable to swallow tablets or capsules. Optionally, a variety of water-compatible preservatives, colorings, flavorings, and viscosity-adjusting agents may be added to a composition of the invention to improve its palatability and patient tolerability.

The pharmaceutical compositions of this invention also can be administered parenterally, in sterile liquid dosage forms. The pharmaceutical composition is preferably made in the form of a dosage unit containing a particular amount of each active ingredient. Formulations for parenteral administration may be in the form of aqueous or buffered isotonic sterile injection solutions. These solutions may be prepared from sterile powders or granules having one or more of the carriers or diluents mentioned for use in the formulations for oral administration. The components may be dissolved in water and/or aqueous solutions. The indicated formulations can contain compatible auxiliaries and excipients, such as anti-oxidants, preservatives, stabilizing agents, emulsifiers, salts for influencing the osmotic pressure, and/or buffer substances.

Other adjuvants and modes of administration are well and widely known in the pharmaceutical art.

Pharmaceutical compositions for use in the treatment methods of the invention may be administered in oral form or by intravenous administration. Oral administration of the therapy is preferred. Dosing for oral administration may be with a regimen calling for single daily dose, or for a single dose every other day, or for multiple, spaced doses throughout the day. The active agents which make up the therapy may be administered simultaneously, either in a combined dosage form or in separate dosage forms intended for substantially simultaneous oral administration. The active agents which make up the therapy may also be administered sequentially, with either active component being administered by a regimen calling for two-step ingestion. Thus, a regimen may call for sequential administration of the active agents with spaced-apart ingestion of the separate, active agents. The time period between the multiple ingestion steps may range from a few minutes to several hours, depending upon the properties of each active agent such a potency, solubility, bioavailability, plasma half-life and kinetic profile of the agent, as well as depending upon the age and condition of the patient. The active agents of the therapy whether administered simultaneously, substantially simultaneously, or sequentially, may involve a regimen calling for administration of one active agent by oral route and the other active agent by intravenous route. Whether the active agents of the therapy are administered by oral or intravenous route, separately or together, each such active agent will be contained in a suitable pharmaceutical formulation of pharmaceutically-acceptable excipients, diluents or other formulations components.

### Clinical Uses of the Combination Therapy of the Invention

The present invention provides methods for the reproducible formulation of a first quantity of a magnesium valproate selected from the group consisting of magnesium valproate hydrate and L-carnitine magnesium valproate, and a second quantity of L-carnitine into pharmaceutical dosage forms using conventional pharmaceutical techniques. In addition, the inventors have shown that aqueous solutions of the invention are readily prepared to have valproate concentrations in the range from about 10 mg/mL to about 150 mg/mL and L-carnitine concentrations in the range from about 25 mg/mL to about 1,000 mg/mL. Aqueous solutions of magnesium valproate hydrate comprise fully ionized solutions of magnesium ions, valproate ions, and L-carnitine which, after parenteral administration to a subject, are completely bioavailable. The inventors have also shown that a composition of the invention is soluble in simulated gastric fluid USP and in simulated intestinal fluid USP. Given this solubility, the inventors believe that a composition of the invention, when administered per os to a subject, will exhibit a valproate bioavailability at least about 90% relative to intravenous infusion, a bioavailability that is equivalent to or exceeds that of divalproex sodium. On these bases, therefore, the inventors believe that a composition of the present invention will be administered to subjects in need of valproate therapy as a therapeutically effective and biologically available substitute for valproic acid, divalproex sodium, valproate sodium, and other valproate salt compositions and who are at risk for L-carnitine deficiency.

For example, the inventors believe that a composition of the present invention will be substituted for valproic acid, divalproex sodium, valproate sodium and other valproate salt compositions in compositions useful for the treatment of neurological disorders as disclosed, by way of example, in U.S. Patent Applications 20050095579, 20050090548, 20050090449, 20050075282, 20050070524, and 20050065340, as well as in U.S. Pat. Nos. 6,406,716, 6,323,236, 6,287,598, and 5,945,416 and in international patents EP 1371366 A1, EP 0966967 A3, EP 1158973 B1, WO 2005070461, WO 2005063297, WO 2005051915, WO 2005049040, and WO 2004101603. Further, the inventors believe that a composition of the present invention will be substituted for valproic acid, divalproex sodium, valproate sodium and other valproate salt compositions in compositions useful for the treatment of immunological disorders as disclosed, by way of example, in U.S. Patent Applications 20050119261, 20050090553, 20050065596, 20050065173, 20050054091, as well as in U.S. Pat. Nos. 5,506,224 and 5,432,176 and in international patents EP 1529527 A1, EP 1293205 A1, EP 1170008 A1, EP 1301184 B1, WO 2005023179, WO 2005018578, WO 2004113305, WO 2004096216, WO 2004096224, and WO 2004050076. Likewise, the inventors believe that a composition of the present invention will be substituted for valproic acid, divalproex sodium, valproate sodium and other valproate salt compositions in compositions useful for the treatment of viral-related disorders as disclosed, by way of example, in the report of Smith [Retrovirology Sep. 19, 2005, 2(1): 56], Cohen [Science Aug. 12, 2005, 309(5737): 999-1000], Lehrman et al. [Lancet Aug. 13, 2005, 366(9485): 549-555], and Ylisastigui et al. [AIDS May 21, 2004, 18(8): 1101-1108].

The following examples present representative compositions of the present invention. The examples are representative of the scope of the invention, and as such are not to be considered or construed as limiting the invention recited in the appended claims.

### Example 1

### Preparation of a Palatable Liquid Dosage Form of the Invention. Method A

About 1,000 mg of magnesium valproate hydrate was accurately weighed and dissolved in 100 mL of purified water USP. 100 mg of magnesium valproate hydrate was added incrementally, and sufficient time was allowed for dissolution to provide a clear and colorless solution. Incremental additions stopped when white solid was observed in the solution, indicating that no additional magnesium valproate hydrate would dissolve. The solubility limit for magnesium valproate hydrate was reached when the valproate concentration of the solution was about 150 mg/mL.

The magnesium valproate solution having a concentration of about 150 mg/mL was divided into 5 mL portions. To each portion was added a known mass of L-carnitine inner salt, accurately weighed. The solutions described in Table 2 were thus obtained. Each solution was tested by two independent analysts.

TABLE 2

| Composition | Appearance | Odor | Taste |
|---|---|---|---|
| 100 mg/mL valproate + 25 mg/mL | Clear, colorless | None | None |
| L-carnitine in water | solution | | |
| 100 mg/mL valproate + 100 mg/mL | Clear, colorless | None | None |
| L-carnitine in water | solution | | |
| 100 mg/mL valproate + 500 mg/mL | Clear, colorless | None | None |
| L-carnitine in water | solution | | |

The experiment was repeated but valproate was added as L-carnitine magnesium valproate. Incremental portions of L-carnitine magnesium valproate were added to purified water until the solubility limit was reached, as indicated by the presence of white solid in the colorless aqueous supernatant. The valproate concentration of the solution was about 150 mg/mL. The solution was divided into portions, and known masses of L-carnitine inner salt, accurately weighed, were added to provide solutions having the compositions shown in Table 2. Each solution was tested by two analysts.

TABLE 3

| Composition | Appearance | Odor | Taste |
|---|---|---|---|
| 100 mg/mL valproate + 25 mg/mL | Clear, colorless | None | None |
| L-carnitine in water | solution | | |
| 100 mg/mL valproate + 100 mg/mL | Clear, colorless | None | None |
| L-carnitine in water | solution | | |
| 100 mg/mL valproate + 500 mg/mL | Clear, colorless | None | None |
| L-carnitine in water | solution | | |

### Example 2

### Preparation of a Palatable Liquid Dosage Form of the Invention. Method B

About 10 g of L-carnitine inner salt was accurately weighed and dissolved in 10 mL of purified water USP to provide a clear and colorless solution. 100 mg portions of magnesium valproate hydrate were added, and the solution was agitated until all material dissolved. A clear and colorless solution was thus obtained. Addition of 100 mg portions of magnesium valproate hydrate continued until white solid remained in the solution and would not dissolve. At the limit of solubility the valproate concentration was about 150 mg/mL and the L-carnitine concentration was about 1,000 mg/mL; the solution was clear and colorless, had no odor, and had no taste. The solution was free-flowing, had a viscosity similar to that of water, and had a near neutral pH.

The experiment was repeated, and 100 mg portions of L-carnitine magnesium valproate were added in place of magnesium valproate hydrate. At the limit of solubility, the valproate concentration was about 150 mg/mL; the solution was clear and colorless, had no odor, and had no taste. The solution was free-flowing, had a viscosity similar to that of water, and had a near neutral pH.

### Example 3

### Sterilization of a Palatable Liquid Dosage Form of the Invention

A palatable liquid dosage form of the invention is divided into portions. Each portion is subjected to sterilization by (a) heating or (b) filtration through a filter membrane having a 1-micron pore size. The portion of the liquid dosage form that is heated exhibits a “fishy” odor characteristic of trimethylamine, indicating that degradation of L-carnitine has occurred. The portion that is filtered aseptically remains clear and colorless, and has no odor or taste.

The following examples present hypothetically useful therapeutic uses of representative pharmaceutical compositions of the present invention and their anticipated outcomes in treating neurological diseases in subjects requiring such treatment. The examples are representative of the scope of the invention, and as such are not to be considered or construed as limiting the invention recited in the appended claims.

### Example 4

### A Palatable Liquid Dosage Form of the Present Invention in the Treatment of Epilepsy

The therapeutic benefit of a palatable liquid dosage form of the present invention is compared with that of sodium valproate in an open, comparative clinical trial in epileptic patients. Patients in the study population exhibit symptoms such as tonic-clonic convulsions, tonic nonfocal convulsions, simple absence seizures, absence attacks associated with generalized convulsions, partial convulsions, West syndrome or Lennox-Gastaut syndrome. The study population has previously been treated with sodium valproate (alone or in combination with other drugs) for a minimum of 6 months. Then treatment is substituted with the liquid dosage form of the invention (alone or combined with other drugs) for 3 months. The following results are expected to be observed following administration of the liquid dosage form of the invention for 3 months. The percentage of patients without convulsions is expected to increase significantly. The number of patients with no convulsions or only occasional convulsions is expected to increase significantly. Patients with generalized nonconvulsive or partial epilepsy are expected to obtain significantly greater therapeutic benefit from treatment with the liquid dosage form of the invention rather than sodium valproate, although patients in all other types of epilepsy are expected to obtain equivalent therapeutic benefit from the liquid dosage form of the invention or sodium valproate treatment. Patients who receive the liquid dosage form of the invention are expected to have fewer side effects (e.g., gastric discomfort or ulceration, malaise) and are expected to exhibit decreased risk for L-carnitine deficiency.

All mentioned references are incorporated by reference as if here written. When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

Without further elaboration, it is believed that one skilled in the art can, using the preceding description, utilize the present invention to its fullest extent. The specific embodiments are, therefore, to be construed as merely illustrative, and not limitative of the remainder of the disclosure in any way whatsoever.

I claim:

1. A palatable liquid valproate composition having quantitatively precise defined proportions of valproate and L-carnitine comprising defined proportions of a pure magnesium valproate and L-carnitine inner salt in water.

2. The liquid valproate composition of claim 1 wherein the pure magnesium valproate is selected from a group consisting of magnesium valproate hydrate and L-carnitine magnesium valproate, the concentration of valproate is in the range from about 1 mg/mL to about 150 mg/mL and the concentration of L-carnitine inner salt is in the range from about 25 mg/mL to about 1,000 mg/mL.

3. The liquid valproate composition of claim 1, wherein said composition is substantially clear.

4. The liquid valproate composition of claim 1, wherein said composition has a neutral pH.

5. The liquid valproate composition of claim 1, wherein said composition is essentially odorless.

6. The liquid valproate composition of claim 1, wherein said composition is essentially tasteless.

7. A liquid dosage form comprising the liquid valproate composition of claim 1 and a flavoring agent.

8. A method of preparing a liquid valproate composition having quantitatively precise defined proportions of valproate and L-carnitine comprising dissolving a first mass of a pure magnesium valproate, wherein the pure magnesium valproate is selected from a group consisting of magnesium valproate hydrate and L-carnitine magnesium valproate, and a second mass of L-carnitine inner salt in a volume of water sufficient to provide a clear and colorless solution.

9. The method of a preparing liquid valproate composition of claim 8 wherein the first mass of the pure magnesium valproate provides a valproate concentration in the range from about 10 mg/mL to about 150 mg/mL and the second mass of L-carnitine inner salt provides an L-carnitine concentration in the range from about 25 mg/mL to about 1,000 mg/mL.

10. A method of treating of a subject comprising providing a liquid dosage form comprising a first therapeutically effective amount of magnesium valproate selected from a group consisting of magnesium valproate hydrate and L-carnitine magnesium valproate, and a second therapeutically effective amount of L-carnitine inner salt in a volume of water sufficient to provide a clear and colorless solution and administering said liquid dosage form to said subject.

11. The method of claim 10, wherein said liquid dosage form is administered from 1 to about 3 times daily.
